(19) United States
(12) Patent Application Publication
(10) Pub. No.: US 2017/0049376 A1
(43) Pub. Date: Feb. 23, 2017

(54) METHODS AND APPARATUSES FOR DETECTING MOTION DISORDER SYMPTOMS BASED ON SENSOR DATA

(71) Applicant: QUALCOMM Incorporated, San Diego, CA (US)

(72) Inventors: Jin Won LEE, San Diego, CA (US); Xinzhou WU, San Diego, CA (US); Parixit Laljibhai AGHERA, San Diego, CA (US); Rashid Ahmed Akbar ATTAR, San Diego, CA (US)

(21) Appl. No.: 15/237,851

(22) Filed: Aug. 16, 2016

Related U.S. Application Data
(60) Provisional application No. 62/206,728, filed on Aug. 18, 2015.

(51) Int. Cl. A61B 5/00; A61B 5/11

(52) U.S. Cl. CPC A61B 5/4082; A61B 5/1123; A61B 5/7278; A61B 5/0022; A61B 5/6824; A61B 5/6898; A61B 5/6829; A61B 5/742; A61B 2562/0219; A61B 2562/0223; A61B 2562/0204

(57) ABSTRACT

Disclosed are techniques for determining a severity of motion disorder symptoms by receiving sensor data from one or more sensors, determining that the sensor data represents one or more activities of daily life (ADLs) of a user, assigning one or more probabilities to the one or more determined ADLs, each probability of the one or more probabilities indicating a confidence level that the sensor data represents a corresponding ADL, and providing the sensor data and the one or more probabilities to a motion disorder symptom scoring module that generates one or more scores for the one or more determined ADLs based on the sensor data, each score of the one or more scores indicating the severity of the motion disorder symptoms for a corresponding ADL, and combines the one or more scores and the one or more probabilities to generate an aggregated severity score for the motion disorder symptoms.

### CROSS-REFERENCE TO RELATED APPLICATIONS

The present Application for Patent claims the benefit of U.S. Provisional Application No. 62/206,728, entitled “METHODS AND APPARATUSES FOR DETECTING MOTION DISORDER SYMPTOMS BASED ON SENSOR DATA,” filed Aug. 18, 2015, assigned to the assignee hereof, and expressly incorporated herein by reference in its entirety.

### INTRODUCTION

Various embodiments described herein relate to detecting motion disorder symptoms based on sensor data.

Mobile communications networks are in the process of offering increasingly sophisticated capabilities associated with the motion sensing of a mobile device. New software applications, such as those related to physical activity, symptom monitoring, prescription administration, and the like, may utilize motion sensors to provide new features and services to consumers. For example, to facilitate dose usage control for a motion disorder (e.g., Parkinson's Disease, Multiple Sclerosis, etc.) patient in an ambulatory setting, various sensors worn by the patient can detect the presence and severity of the patient's symptoms. Because the dosage of the patient's medication depends on the detected symptoms, it is important to detect those symptoms accurately. However, activities of daily life (ADLs) that the patient is engaged in can be confused with motion disorder symptoms and/or make it difficult to accurately quantify the severity of the symptoms.

### SUMMARY

The following presents a simplified summary relating to one or more aspects and/or embodiments associated with the mechanisms disclosed herein for detecting motion disorder symptoms based on sensor data. As such, the following summary should not be considered an extensive overview relating to all contemplated aspects and/or embodiments, nor should the following summary be regarded to identify key or critical elements relating to all contemplated aspects and/or embodiments or to delineate the scope associated with any particular aspect and/or embodiment. Accordingly, the following summary has the sole purpose to present certain concepts relating to one or more aspects and/or embodiments relating to the mechanisms disclosed herein in a simplified form to precede the detailed description presented below.

A method for determining a severity of motion disorder symptoms based on sensor data includes receiving, at a processor of a mobile device, the sensor data from one or more sensors, determining, by the processor, that the sensor data represents one or more activities of daily life (ADLs) of a user, assigning, by the processor, one or more probabilities to the one or more determined ADLs, each probability of the one or more probabilities indicating a confidence level that the sensor data represents a corresponding ADL of the one or more determined ADLs, and providing, by the processor, the sensor data and the one or more probabilities to a motion disorder symptom scoring module, wherein the motion disorder symptom scoring module generates one or more scores for the one or more determined ADLs based on the sensor data, each score of the one or more scores indicating the severity of the motion disorder symptoms for a corresponding ADL of the one or more determined ADLs, and combines the one or more scores and the one or more probabilities to generate an aggregated severity score for the motion disorder symptoms.

A method of operating a device that is configured to execute a motion disorder symptom scoring module includes receiving, at the motion disorder symptom scoring module, sensor data that is representative of one or more ADLs of a user, receiving, at the motion disorder symptom scoring module, one or more probabilities that are assigned to the one or more determined ADLs, each probability of the one or more probabilities indicating a confidence level that the sensor data represents a corresponding ADL of the one or more determined ADLs, generating, by the motion disorder symptom scoring module, one or more scores for the one or more determined ADLs based on the sensor data, each score of the one or more scores indicating the severity of the motion disorder symptoms for a corresponding ADL of the one or more determined ADLs, and combining, by the motion disorder symptom scoring module, the one or more scores and the one or more probabilities to generate an aggregated severity score for the motion disorder symptoms.

An apparatus for determining a severity of motion disorder symptoms based on sensor data includes at least one processor configured to: receive the sensor data from one or more sensors, determine that the sensor data represents one or more ADLs of a user, assign one or more probabilities to the one or more determined ADLs, each probability of the one or more probabilities indicating a confidence level that the sensor data represents a corresponding ADL of the one or more determined ADLs, and provide the sensor data and the one or more probabilities to a motion disorder symptom scoring module, wherein the motion disorder symptom scoring module is configured to generate one or more scores for the one or more determined ADLs based on the sensor data, each score of the one or more scores indicating the severity of the motion disorder symptoms for a corresponding ADL of the one or more determined ADLs, and combine the one or more scores and the one or more probabilities to generate an aggregated severity score for the motion disorder symptoms.

An apparatus for operating a device that is configured to execute a motion disorder symptom scoring module includes at least one processor configured to: receive sensor data that is representative of one or more ADLs of a user, receive one or more probabilities that are assigned to the one or more determined ADLs, each probability of the one or more probabilities indicating a confidence level that the sensor data represents a corresponding ADL of the one or more determined ADLs, generate one or more scores for the one or more determined ADLs based on the sensor data, each score of the one or more scores indicating the severity of the motion disorder symptoms for a corresponding ADL of the one or more determined ADLs, and combine the one or more scores and the one or more probabilities to generate an aggregated severity score for the motion disorder symptoms.

A non-transitory computer-readable medium for determining a severity of motion disorder symptoms based on sensor data includes at least one instruction to cause a processor of a mobile device to receive the sensor data from one or more sensors, at least one instruction to cause the processor to determine that the sensor data represents one or more ADLs of a user, at least one instruction to cause the processor to assign one or more probabilities to the one or more determined ADLs, each probability of the one or more probabilities indicating a confidence level that the sensor data represents a corresponding ADL of the one or more determined ADLs, and at least one instruction to cause the processor to provide the sensor data and the one or more probabilities to a motion disorder symptom scoring module, wherein the motion disorder symptom scoring module generates one or more scores for the one or more determined ADLs based on the sensor data, each score of the one or more scores indicating the severity of the motion disorder symptoms for a corresponding ADL of the one or more determined ADLs, and combines the one or more scores and the one or more probabilities to generate an aggregated severity score for the motion disorder symptoms.

A non-transitory computer-readable medium for operating a device that is configured to execute a motion disorder symptom scoring module includes at least one instruction to cause the motion disorder symptom scoring module to receive sensor data that is representative of one or more ADLs of a user, at least one instruction to cause the motion disorder symptom scoring module to receive one or more probabilities that are assigned to the one or more determined ADLs, each probability of the one or more probabilities indicating a confidence level that the sensor data represents a corresponding ADL of the one or more determined ADLs, at least one instruction to cause the motion disorder symptom scoring module to generate one or more scores for the one or more determined ADLs based on the sensor data, each score of the one or more scores indicating the severity of the motion disorder symptoms for a corresponding ADL of the one or more determined ADLs, and at least one instruction to cause the motion disorder symptom scoring module to combine the one or more scores and the one or more probabilities to generate an aggregated severity score for the motion disorder symptoms.

Other objects and advantages associated with the mechanisms disclosed herein will be apparent to those skilled in the art based on the accompanying drawings and detailed description.

### BRIEF DESCRIPTION OF THE DRAWINGS

A more complete appreciation of aspects of the disclosure and many of the attendant advantages thereof will be readily obtained as the same becomes better understood by reference to the following detailed description when considered in connection with the accompanying drawings which are presented solely for illustration and not limitation of the disclosure, and in which:

FIG. 1 illustrates an exemplary operating environment for a mobile device in communication with various sensors, according to at least one aspect of the disclosure.

FIG. 2 illustrates an exemplary mobile device that may be used in an operating environment for detecting motion disorder symptoms based on sensor data, according to one aspect of the disclosure.

FIG. 3 illustrates an exemplary server according to various aspects of the disclosure.

FIG. 4 illustrates an exemplary process for classifying activities of daily living (ADLs) and estimating a severity score of symptoms of a motion disorder.

FIG. 5 illustrates an exemplary flow for determining a severity of motion disorder symptoms based on sensor data, according to at least one aspect of the disclosure.

FIG. 6 illustrates an exemplary flow for operating a device configured to execute a motion disorder symptom scoring module according to at least one aspect of the disclosure.

FIGS. 7-8 are simplified block diagrams of several sample aspects of apparatuses configured to determine a severity of motion disorder symptoms as taught herein.

### DETAILED DESCRIPTION

Disclosed are methods and systems for determining a severity of motion disorder symptoms based on sensor data. In an aspect, a processor of a mobile device receives the sensor data from one or more sensors, determines that the sensor data represents one or more activities of daily life (ADLs) of a user, assigns one or more probabilities to the one or more determined ADLs, each probability of the one or more probabilities indicating a confidence level that the sensor data represents a corresponding ADL of the one or more determined ADLs, and provides the sensor data and the one or more probabilities to a motion disorder symptom scoring module. The motion disorder symptom scoring module generates one or more scores for the one or more determined ADLs based on the sensor data, each score of the one or more scores indicating the severity of the motion disorder symptoms for a corresponding ADL of the one or more determined ADLs, and combines the one or more scores and the one or more probabilities to generate an aggregated severity score for the motion disorder symptoms.

In an aspect, a motion disorder symptom scoring module receives sensor data that is representative of one or more ADLs of a user, receives one or more probabilities that are assigned to the one or more determined ADLs, each probability of the one or more probabilities indicating a confidence level that the sensor data represents a corresponding ADL of the one or more determined ADLs, generates one or more scores for the one or more determined ADLs based on the sensor data, each score of the one or more scores indicating the severity of the motion disorder symptoms for a corresponding ADL of the one or more determined ADLs, and combines the one or more scores and the one or more probabilities to generate an aggregated severity score for the motion disorder symptoms.

Various aspects are disclosed in the following description and related drawings. Alternate aspects may be devised without departing from the scope of the disclosure. Additionally, well-known elements of the disclosure will not be described in detail or will be omitted so as not to obscure the relevant details of the disclosure.

The words “exemplary” and/or “example” are used herein to mean “serving as an example, instance, or illustration.” Any aspect described herein as “exemplary” and/or “example” is not necessarily to be construed as preferred or advantageous over other aspects. Likewise, the term “aspects of the disclosure” does not require that all aspects of the disclosure include the discussed feature, advantage or mode of operation.

The terminology used herein is for the purpose of describing particular embodiments only and not to limit any embodiments disclosed herein. As used herein, the singular forms “a”, “an” and “the” are intended to include the plural forms as well, unless the context clearly indicates otherwise. It will be further understood that the terms “comprises”, “comprising”, “includes” and/or “including”, when used herein, specify the presence of stated features, integers, steps, operations, elements, and/or components, but do not preclude the presence or addition of one or more other features, integers, steps, operations, elements, components, and/or groups thereof.

Further, many aspects are described in terms of sequences of actions to be performed by, for example, elements of a computing device. It will be recognized that various actions described herein can be performed by specific circuits (e.g., an application specific integrated circuit (ASIC)), by program instructions being executed by one or more processors, or by a combination of both. Additionally, these sequence of actions described herein can be considered to be embodied entirely within any form of computer-readable storage medium having stored therein a corresponding set of computer instructions that upon execution would cause an associated processor to perform the functionality described herein. Thus, the various aspects of the disclosure may be embodied in a number of different forms, all of which have been contemplated to be within the scope of the claimed subject matter. In addition, for each of the aspects described herein, the corresponding form of any such aspects may be described herein as, for example, “logic configured to” perform the described action.

As noted above, mobile communications networks are in the process of offering increasingly sophisticated capabilities associated with the motion sensing of a mobile device. New software applications, such as those related to physical activity, symptom monitoring, prescription administration, and the like, may utilize motion sensors to provide new features and services to consumers. For example, to facilitate dose usage control for a user with a motion disorder (e.g., Parkinson's Disease, Multiple Sclerosis, etc.), the user can wear various sensors that can detect the presence and severity of the user's symptoms.

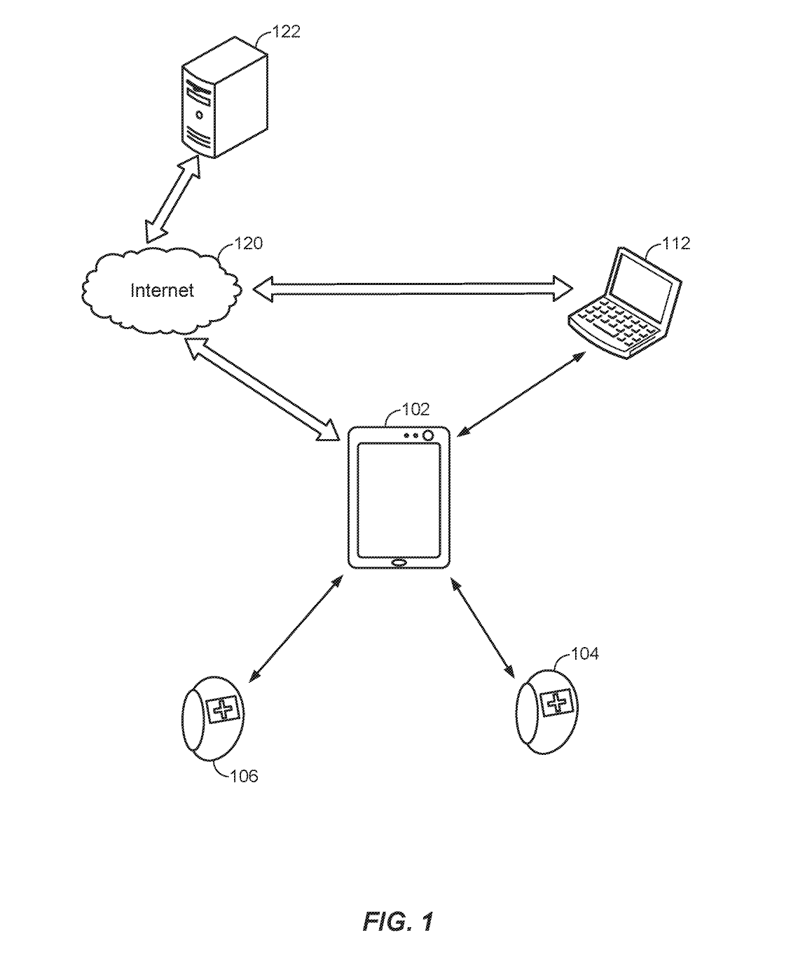

FIG. 1 illustrates an exemplary operating environment for a mobile device 102 in communication with various external wearable sensors according to at least one aspect of the disclosure. Specifically, in the example of FIG. 1, the mobile device 102 may belong to a user with a motion disorder and be in communication with a wrist sensor 104 and an ankle sensor 106 worn by the user. The mobile device 102 may communicate with the wrist sensor 104 and the ankle sensor 106 via any suitable wireless network, such as Wi-Fi, Bluetooth®, LTE-Direct, etc. Each of the mobile device 102, the wrist sensor 104, and the ankle sensor 106 may include various motion sensors, such as one or more accelerometers, one or more gyroscopes, one or more magnetometers, one or more microphones, etc. Note that although FIG. 1 illustrates only two wearable sensors, there may be any number of wearable sensors in communication with the mobile device 102.

The mobile device 102 may also be in communication with a local personal computer, such as laptop computer 112, which may belong to the same user or a third-party monitoring the health of the user. Additionally, the mobile device 102 may be able to connect to the Internet 120 in various ways, such as over a cellular network, a Wi-Fi or other wireless local area network (WLAN), etc. The mobile device 102 may also be able to communicate with one or more third-party servers 122 via the Internet 120.

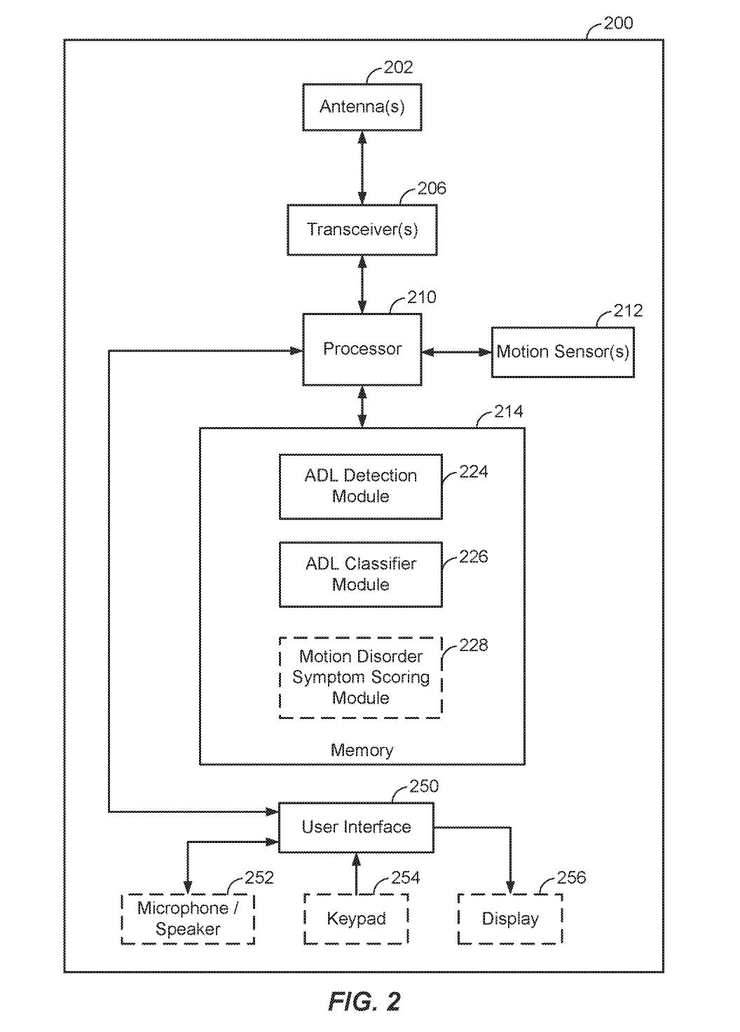

FIG. 2 is a block diagram illustrating various components of an exemplary mobile device 200. Depending on the embodiment, the mobile device 200 may correspond to the mobile device 102, the wrist sensor 104, or the ankle sensor 106 in FIG. 1. For the sake of simplicity, the various features and functions illustrated in the box diagram of FIG. 2 are connected together using a common bus that is meant to represent that these various features and functions are operatively coupled together. Those skilled in the art will recognize that other connections, mechanisms, features, functions, or the like, may be provided and adapted as necessary to operatively couple and configure an actual portable wireless device. Further, it is also recognized that one or more of the features or functions illustrated in the example of FIG. 2 may be further subdivided or two or more of the features or functions illustrated in FIG. 2 may be combined.

The mobile device 200 may include one or more transceiver(s) 206 that may be connected to one or more antennas 202. Where the mobile device 200 corresponds to the mobile device 102, at least one of the one or more transceivers 206 may comprise suitable devices, hardware, and/or software for communicating with and/or detecting signals to/from the wrist sensor 104, the ankle sensor 106, and/or the laptop computer 112. Likewise, at least one of the one or more transceivers 206 may comprise suitable devices, hardware, and/or software for communicating over the Internet 120. Alternatively, where the mobile device 200 corresponds to one or more of the wrist sensor 104 and the ankle sensor 106, at least one of the one or more transceivers 206 may comprise suitable devices, hardware, and/or software for communicating with and/or detecting signals to/from the mobile device 102.

One or more motion sensors 212 may be coupled to a processor 210 to provide movement and/or orientation information of the mobile device 200. By way of example, the one or more motion sensors 212 may utilize an accelerometer (e.g., a microelectromechanical system (MEMS) device), a gyroscope, a geomagnetic sensor (e.g., a compass), an altimeter (e.g., a barometric pressure altimeter), and/or any other type of movement detection sensor. Moreover, the one or more motion sensors 212 may include a plurality of different types of devices and combine their outputs in order to provide motion information. For example, the one or more motion sensors 212 may use a combination of a multi-axis accelerometer and orientation sensors to provide the ability to compute positions in two-dimensional and/or three-dimensional coordinate systems.

The processor 210 may include one or more microprocessors, microcontrollers, and/or digital signal processors that provide processing functions, as well as other calculation and control functionality. The processor 210 may also include memory 214 for storing data and software instructions for executing programmed functionality within the mobile device 200. The memory 214 may be on-board the processor 210 (e.g., within the same integrated circuit (IC) package), and/or the memory 214 may be external memory to the processor 210 and functionally coupled over a data bus. The functional details associated with aspects of the disclosure will be discussed in more detail below.

A number of software modules and data tables may reside in memory 214 and be utilized by the processor 210 in order to manage both communications and the detection of motion disorder symptoms based on sensor data. As illustrated in FIG. 2, memory 214 may include an ADL detection module 224, an ADL classifier module 226, and an optional motion disorder symptom scoring module 228. The motion disorder symptom scoring module 228 is optional because, as will be described below, while it may reside on the mobile device 200, it may alternatively reside on a third-party device, such as laptop computer 112 and/or server 122, or, where the mobile device 200 corresponds to one of the wrist sensor 104 or the ankle sensor 106, on the mobile device 102.

It should be appreciated that the organization of the memory 214 contents as shown in FIG. 2 is merely exemplary, and as such the functionality of the modules and/or data structures may be combined, separated, and/or be structured in different ways depending upon the implementation of the mobile device 200. For example, while in this example the ADL detection module 224, the ADL classifier module 226, and the optional motion disorder symptom scoring module 228 are illustrated as being separate features, it should be recognized that such procedures may be combined together as one procedure or perhaps with other procedures, or otherwise further divided into a plurality of sub-procedures. Further, while the modules shown in FIG. 2 are illustrated in the example as being contained in the memory 214, it should be recognized that in certain implementations, such procedures may be provided for or otherwise operatively arranged using other or additional mechanisms. For example, all or part of the ADL detection module 224, the ADL classifier module 226, and/or the optional motion disorder symptom scoring module 228 may be provided in firmware or as logic circuits within the processor 210.

The mobile device 200 may further include a user interface 250 that provides any suitable interface systems, such as a microphone/speaker 252, keypad 254, and display 256 that allows user interaction with the mobile device 200. The microphone/speaker 252 provides for voice communication services. The keypad 254 comprises any suitable buttons for user input. The display 256 comprises any suitable display, such as, for example, a backlit liquid crystal display (LCD), and may further include a touch screen display for additional user input modes. As shown in FIG. 2, the microphone/speaker 252, keypad 254, and display 256 are optional, since when the mobile device 200 corresponds to one of the wrist sensor 104 or the ankle sensor 106, the mobile device 200 may not include one or more of the microphone/speaker 252, keypad 254, and display 256.

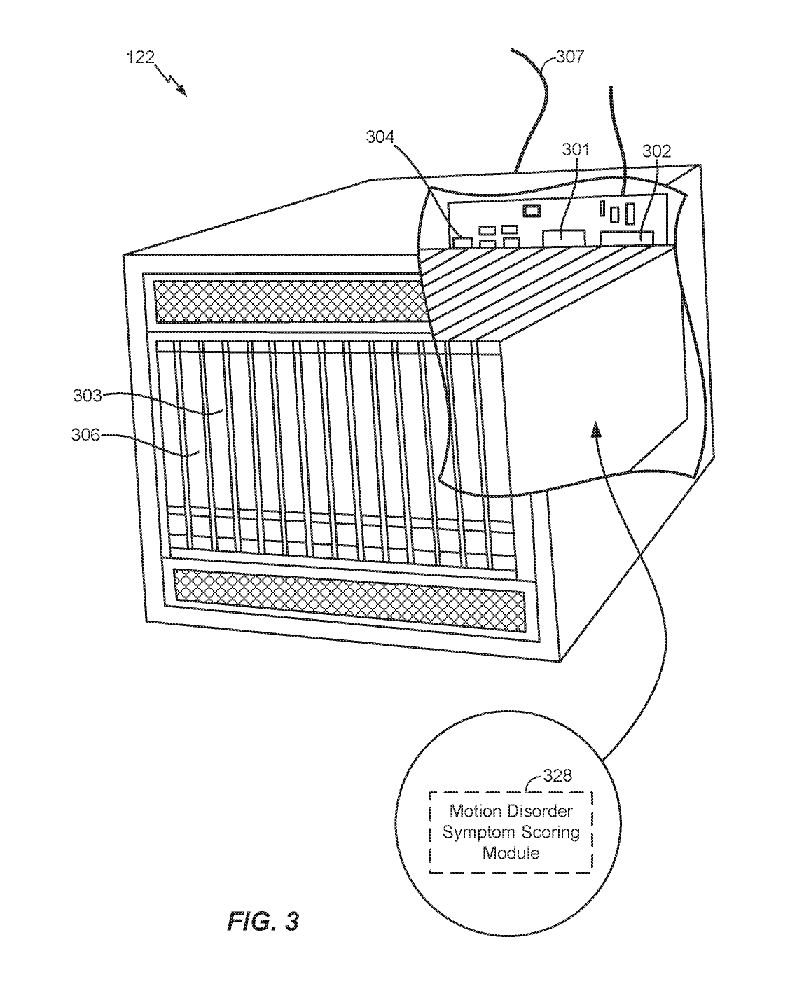

The various embodiments may be implemented at least partially on any of a variety of commercially available server devices, such as server 122 illustrated in FIG. 3. In FIG. 3, the server 122 includes a processor 301 coupled to volatile memory 302 and a large capacity nonvolatile memory 303, such as a disk drive. The server 122 may also include a floppy disc drive, compact disc (CD) or digital video disc (DVD) disc drive 306 coupled to the processor 301. The server 122 may also include network access ports 304 coupled to the processor 301 for establishing data connections with a network 307, such as a local area network coupled to other broadcast system computers and servers or to the Internet 120.

In an embodiment, the large capacity nonvolatile memory 303 may include an optional motion disorder symptom scoring module 328. The motion disorder symptom scoring module 328 is optional because, while it may reside on the server 122, it may alternatively reside on the mobile device 102. Note that although the optional motion disorder symptom scoring module 328 is illustrated as being an executable module stored in the large capacity nonvolatile memory 303, it may be provided in firmware or as a logic circuit within the processor 301.

As noted above, given a user with a motion disorder, because the dosage of the user's medication depends on the detected symptoms, it is important to detect those symptoms accurately. However, ADLs that the user is engaged in can be confused with motion disorder symptoms and/or make it difficult to accurately quantify the severity of the symptoms. Accordingly, the present disclosure provides a method and apparatus for more accurately detecting motion disorder symptoms based on sensor data.

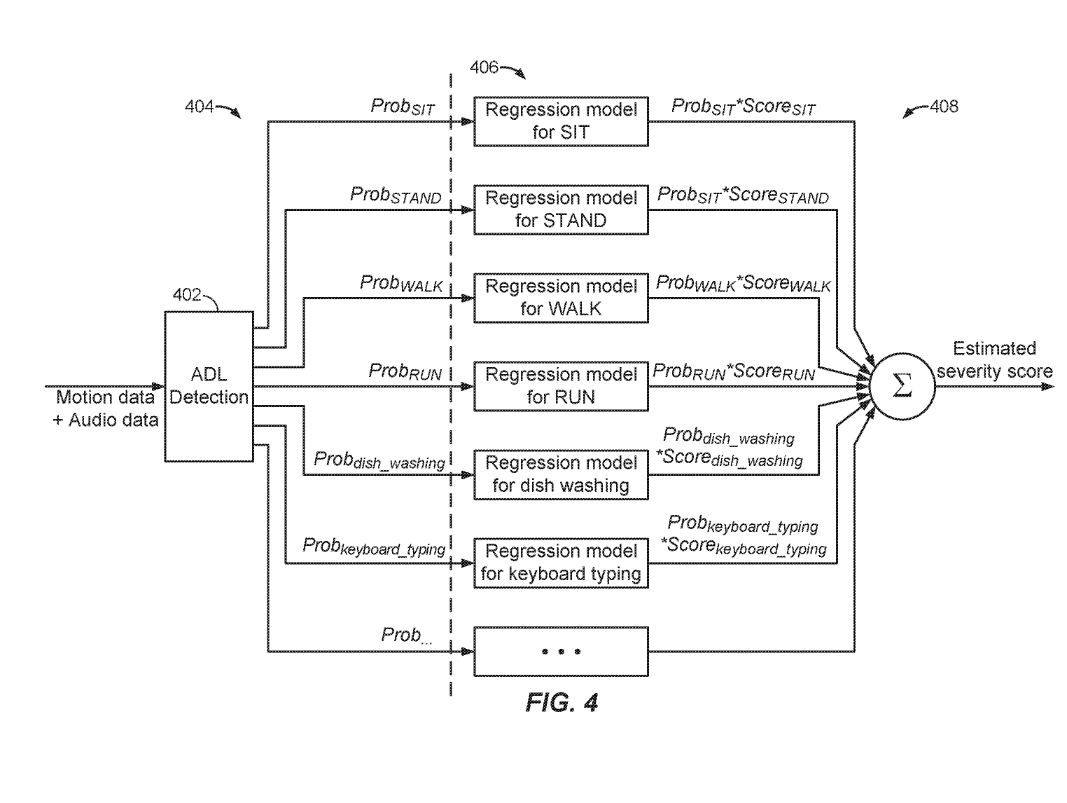

FIG. 4 illustrates an exemplary process for classifying ADLs and estimating a severity score of symptoms of a motion disorder. At 402, the operation of detecting ADLs of a user is performed based on motion data and audio data from one or more sensors. With reference to FIG. 2, where the mobile device 200 corresponds to the mobile device 102 in FIG. 1, the ADL detection module 224 may determine whether or not motion and audio data from the wrist sensor 104 and the ankle sensor 106 represents one or more ADLs of the user. Alternatively, where the mobile device 200 corresponds to one of the wrist sensor 104 or the ankle sensor 106, the ADL detection module 224 may determine whether or not motion from the one or more motion sensors 212 and audio data from the microphone 252 represents one or more ADLs of the user.

In an embodiment, only ADLs that are relevant to the user's motion disorder may be detected. Regarding which ADLs to detect, there are three criteria indicating the relevance of the ADL to the user's motion disorder:

1. Activities that are of statistical importance

2. Activities that last more than five minutes when they occur, for example, dyskinesia lasts for at least 10 minutes when it occurs

3. Activities that can cause confusion with tremors or dyskinesia

Examples of activities that fit all three criteria include walking (relevant to dyskinesia) and driving (relevant to tremors). Examples of activities that fit two criteria include dish washing, typing on a keyboard, and possibly dressing. Examples of activities that fit one criterion include drinking, folding laundry, cutting food, combing hair, and bagging groceries.

At 404, the operation of classifying the detected ADLs is performed. With reference to FIG. 2, the ADL classifier module 226 may classify the ADLs detected by the ADL detection module 224. There may be any number of categories of ADLs into which the detected ADLs can be classified. For example, ADLs may be classified as SIT, STAND, WALK, RUN, DISH_WASHING, KEYBOARD_TYPING, and the like. At a high level, the ADL classifier module 226 analyzes the data from the wrist sensor 104 and the ankle sensor 106 that has been determined to represent an ADL by the ADL detection module 224 and determines what type of ADL the sensor data represents, and with what probability/likelihood the data represents that ADL. The ADL classifier module 226 outputs the probability that the detected ADLs are particular types of ADLs, represented in FIG. 4 as, for example, Prob_{SIT}, Prob_{STAND}, Prob_{WALK}, Prob_{RUN}, etc.

At 406, a regression analysis is performed on the classified ADLs to calculate a severity score of the user's motion disorder symptoms. With reference to FIG. 2, the motion disorder symptom scoring model 228 may calculate a severity score for the ADLs classified by the ADL classifier module 226, represented in FIG. 4 as, for example, Score_{SIT}, Score_{STAND}, Score_{WALK}, Score_{RUN}, etc. Each score is calculated by performing a separate regression analysis for each type of ADL classified at 404. For example, as illustrated in FIG. 4, a separate regression analysis is performed for ADLs classified as SIT, a separate regression analysis is performed for ADLs classified as STAND, and so on. By using a regression model specific to the class of ADL, the motion disorder symptom scoring model 228 can more accurately calculate the severity of the user's motion disorder symptoms. The calculated severity score is combined with the probability that the classification of the ADL is correct, represented in FIG. 4 as, for example, Prob_{SIT}* Score_{SIT}.

At 408, based on the combination of the severity score of the motion disorder symptoms and the probability that the classification of the ADL is correct, an estimated severity score of the user's motion disorder is calculated. Referring to FIG. 2, the motion disorder symptom scoring model 228 (or the motion disorder symptom scoring module 328 in FIG. 3) may estimate the severity score based, in an embodiment, on various rules correlating the severity of symptoms to the severity of the motion disorder.

Based on the determined severity, the amount of the user's medication(s) can be adjusted. In an embodiment, the user may be instructed to change his or her medication via the user interface of the mobile device 102 or the laptop computer 112. Alternatively, the user may be contacted by medical staff personnel with the instructions, and if necessary, an updated prescription.

As noted above, the motion disorder symptom scoring model 228 is an optional component of the mobile device 200. In an embodiment, where the mobile device 200 corresponds to the mobile device 102, rather than be a component of the mobile device 200, it may be a component of the laptop computer 112 or the server 122, where the laptop computer 112 and/or the server 122 belong to medical personnel or a medical facility responsible for the user's care. In another embodiment, where the mobile device 200 corresponds to one of the wrist sensor 104 or the ankle sensor 106, rather than be a component of the mobile device 200, the motion disorder symptom scoring model 228 may be a component of the mobile device 102.

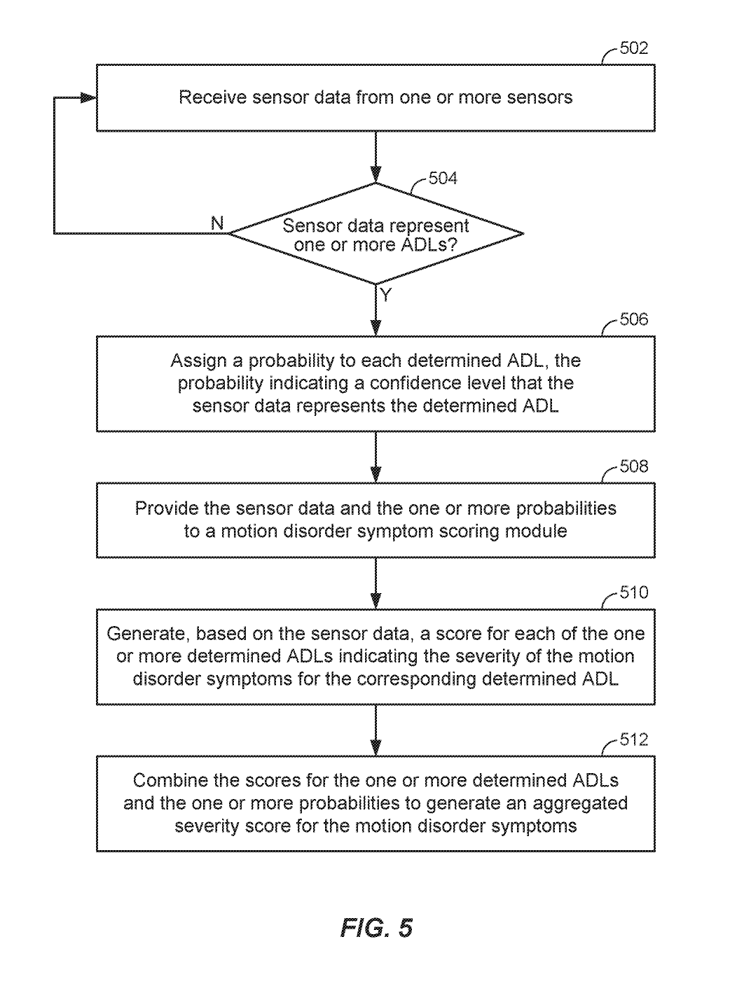

FIG. 5 illustrates an exemplary flow for determining a severity of motion disorder symptoms based on sensor data, according to at least one aspect of the disclosure. The flow illustrated in FIG. 5 may be performed, at least in part, by the mobile device 200 in FIG. 2.

At 502, the mobile device 200, for example, the processor 210 via the transceiver 206, may receive the sensor data from one or more sensors, such as the wrist sensor 104 and/or the ankle sensor 106. Alternatively, where the mobile device 200 corresponds to the wrist sensor 104 or the ankle sensor 106, the processor 210 may receive the sensor data from the one or more motion sensors 212 and/or the microphone 252.

At 504, the mobile device 200, for example, the processor 210, determines whether the sensor data represents one or more ADLs of the user, as described above with reference to 402 of FIG. 4. If it does not, the flow returns to 502. Otherwise, the flow proceeds to 506.

At 506, the mobile device 200, for example, the processor 210, assigns one or more probabilities to the one or more determined ADLs, as described above with reference to 404 of FIG. 4. As discussed above, each probability of the one or more probabilities indicates a confidence level that the sensor data represents a corresponding ADL of the one or more determined ADLs.

At 508, the mobile device 200, for example, the processor 210, provides the sensor data and the one or more probabilities to a motion disorder symptom scoring module, such as the motion disorder symptom scoring module 228/328. As discussed above, where the mobile device 200 corresponds to the mobile device 102, the motion disorder symptom scoring module 228 may be a component of the mobile device 102, the laptop computer 112, or the server 122. Alternatively, where the mobile device 200 corresponds to the wrist sensor 104 or the ankle sensor 106, the motion disorder symptom scoring module 228 may be a component of the mobile device 102. As another alternative, the mobile device 200, for example, the transceiver 206, may provide the sensor data and the one or more probabilities to the motion disorder symptom scoring module 328 on the server 300.

At 510, the motion disorder symptom scoring module 228/328 generates one or more scores for the one or more determined ADLs based on the sensor data, as described above with reference to 406 of FIG. 4. Each score of the one or more scores may indicate the severity of the motion disorder symptoms for a corresponding ADL of the one or more determined ADLs.

At 512, the motion disorder symptom scoring module 228/328 combines the one or more scores and the one or more probabilities to generate an aggregated severity score for the motion disorder symptoms, as described above with reference to 408 of FIG. 4.

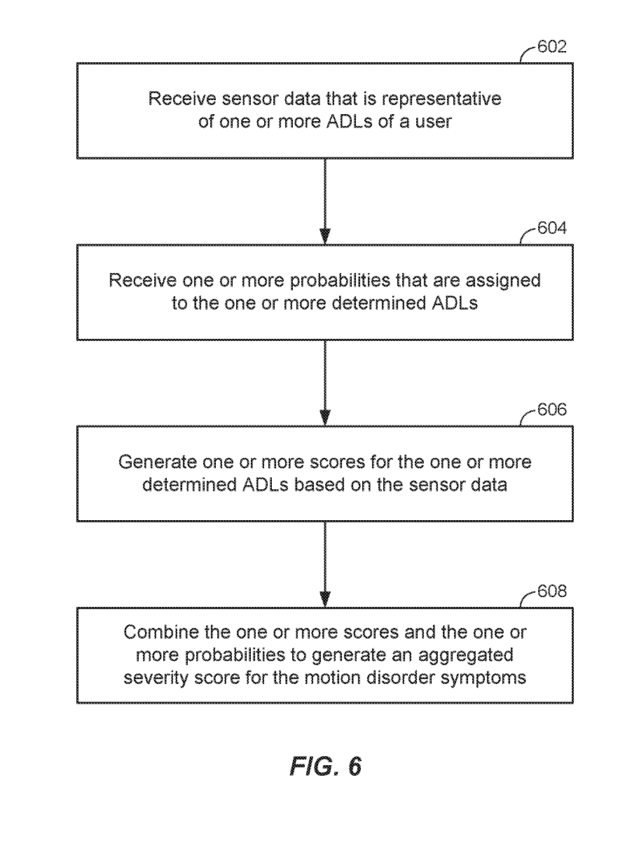

FIG. 6 illustrates an exemplary flow for operating a device that is configured to execute a motion disorder symptom scoring module, such as the motion disorder symptom scoring module 228/328, according to at least one aspect of the disclosure.

At 602, the motion disorder symptom scoring module 228/328 receives sensor data that is representative of one or more ADLs of a user. The motion disorder symptom scoring module 228/328, via the transceiver 206 or the network access ports 304 as applicable, may receive the sensor data from one or more sensors, such as the wrist sensor 104 and/or the ankle sensor 106, or from the mobile device 200.

At 604, the motion disorder symptom scoring module 228/328 receives one or more probabilities that are assigned to the one or more determined ADLs, each probability of the one or more probabilities indicating a confidence level that the sensor data represents a corresponding ADL of the one or more determined ADLs. The motion disorder symptom scoring module 228/328, via the transceiver 206 or the network access ports 304 as applicable, may receive the one or more probabilities from one or more sensors, such as the wrist sensor 104 and/or the ankle sensor 106, or from the mobile device 200.

At 606, the motion disorder symptom scoring module 228/328 generates one or more scores for the one or more determined ADLs based on the sensor data, as described above with reference to 406 of FIG. 4. Each score of the one or more scores indicates the severity of the motion disorder symptoms for a corresponding ADL of the one or more determined ADLs.

At 608, the motion disorder symptom scoring module 228/328 combines the one or more scores and the one or more probabilities to generate an aggregated severity score for the motion disorder symptoms, as described above with reference to 408 of FIG. 4.

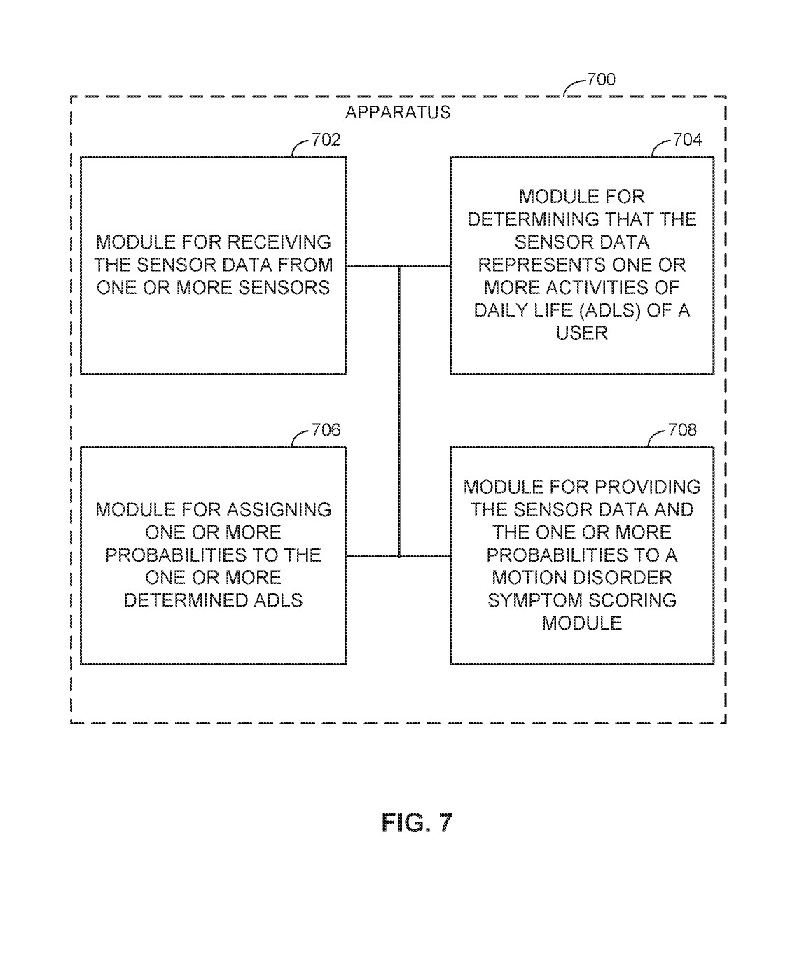

FIG. 7 illustrates an example apparatus 700, such as a mobile device, represented as a series of interrelated functional modules. A module for receiving 702 may correspond at least in some aspects to, for example, a processing system, such as processor 210 in FIG. 2, as discussed herein. A module for determining 704 may correspond at least in some aspects to, for example, a processing system, such as processor 210 in FIG. 2, as discussed herein. A module for assigning 706 may correspond at least in some aspects to, for example, a processing system, such as processor 210 in FIG. 2, as discussed herein. A module for providing 708 may correspond at least in some aspects to, for example, a processing system, such as processor 210 in FIG. 2, as discussed herein.

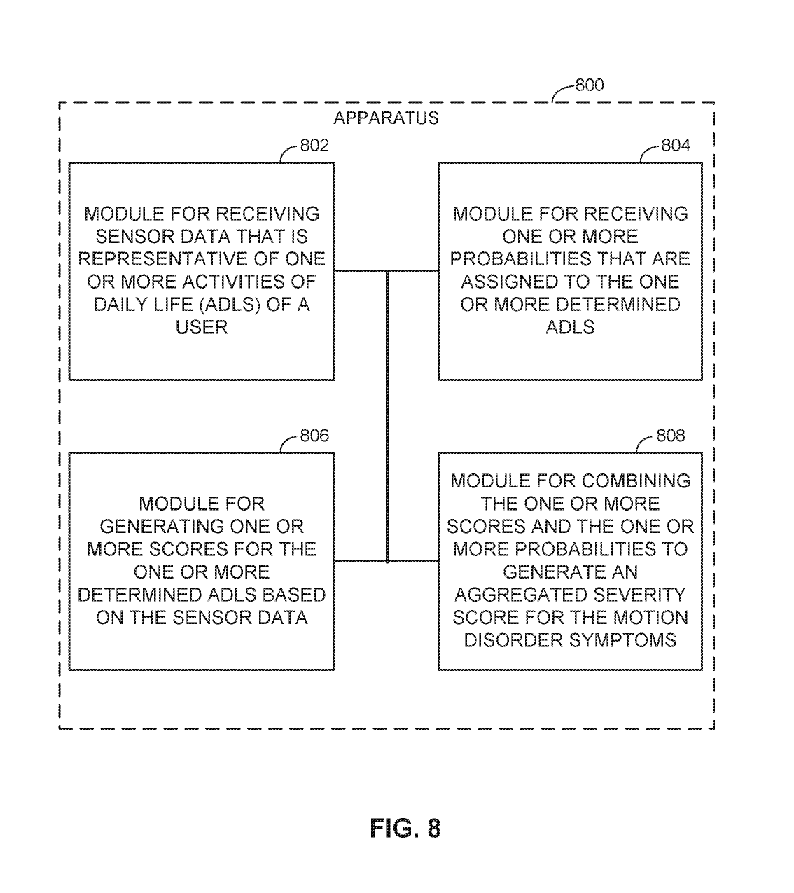

FIG. 8 illustrates an example apparatus 800, such as a mobile device or server having a motion disorder symptom scoring module, represented as a series of interrelated functional modules. A module for receiving 802 may correspond at least in some aspects to, for example, a processing system, such as processor 210 in conjunction with motion disorder symptom scoring module 228 in FIG. 2 or processor 301 in conjunction with motion disorder symptom scoring module 328 in FIG. 3, as discussed herein. A module for receiving 804 may correspond at least in some aspects to, for example, a processing system, such as processor 210 in conjunction with motion disorder symptom scoring module 228 in FIG. 2 or processor 301 in conjunction with motion disorder symptom scoring module 328 in FIG. 3, as discussed herein. A module for generating 806 may correspond at least in some aspects to, for example, a processing system, such as processor 210 in conjunction with motion disorder symptom scoring module 228 in FIG. 2 or processor 301 in conjunction with motion disorder symptom scoring module 328 in FIG. 3, as discussed herein. A module for combining 808 may correspond at least in some aspects to, for example, a processing system, such as processor 210 in conjunction with motion disorder symptom scoring module 228 in FIG. 2 or processor 301 in conjunction with motion disorder symptom scoring module 328 in FIG. 3, as discussed herein.

The functionality of the modules of FIGS. 7-8 may be implemented in various ways consistent with the teachings herein. In some designs, the functionality of these modules may be implemented as one or more electrical components. In some designs, the functionality of these blocks may be implemented as a processing system including one or more processor components. In some designs, the functionality of these modules may be implemented using, for example, at least a portion of one or more integrated circuits (e.g., an ASIC). As discussed herein, an integrated circuit may include a processor, software, other related components, or some combination thereof Thus, the functionality of different modules may be implemented, for example, as different subsets of an integrated circuit, as different subsets of a set of software modules, or a combination thereof Also, it will be appreciated that a given subset (e.g., of an integrated circuit and/or of a set of software modules) may provide at least a portion of the functionality for more than one module.

In addition, the components and functions represented by FIGS. 7-8, as well as other components and functions described herein, may be implemented using any suitable means. Such means also may be implemented, at least in part, using corresponding structure as taught herein. For example, the components described above in conjunction with the “module for” components of FIGS. 7-8 also may correspond to similarly designated “means for” functionality. Thus, in some aspects one or more of such means may be implemented using one or more of processor components, integrated circuits, or other suitable structure as taught herein.

Those of skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Further, those of skill in the art will appreciate that the various illustrative logical blocks, modules, circuits, and algorithm steps described in connection with the aspects disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted to depart from the scope of the present disclosure.

The various illustrative logical blocks, modules, and circuits described in connection with the aspects disclosed herein may be implemented or performed with a general purpose processor, a DSP, an ASIC, an FPGA or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices (e.g., a combination of a DSP and a microprocessor, a plurality of microprocessors, one or more microprocessors in conjunction with a DSP core, or any other such configuration).

The methods, sequences and/or algorithms described in connection with the aspects disclosed herein may be embodied directly in hardware, in a software module executed by a processor, or in a combination of the two. A software module may reside in RAM, flash memory, ROM, EPROM, EEPROM, registers, hard disk, a removable disk, a CD-ROM, or any other form of storage medium known in the art. An exemplary storage medium is coupled to the processor such that the processor can read information from, and write information to, the storage medium. In the alternative, the storage medium may be integral to the processor. The processor and the storage medium may reside in an ASIC. The ASIC may reside in an IoT device. In the alternative, the processor and the storage medium may reside as discrete components in a user terminal.

In one or more exemplary aspects, the functions described may be implemented in hardware, software, firmware, or any combination thereof If implemented in software, the functions may be stored on or transmitted over as one or more instructions or code on a computer-readable medium. Computer-readable media includes both computer storage media and communication media including any medium that facilitates transfer of a computer program from one place to another. A storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Also, any connection is properly termed a computer-readable medium. For example, if the software is transmitted from a website, server, or other remote source using a coaxial cable, fiber optic cable, twisted pair, DSL, or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes CD, laser disc, optical disc, DVD, floppy disk and Blu-ray disc where disks usually reproduce data magnetically and/or optically with lasers. Combinations of the above should also be included within the scope of computer-readable media.

While the foregoing disclosure shows illustrative aspects of the disclosure, it should be noted that various changes and modifications could be made herein without departing from the scope of the disclosure as defined by the appended claims. The functions, steps and/or actions of the method claims in accordance with the aspects of the disclosure described herein need not be performed in any particular order. Furthermore, although elements of the disclosure may be described or claimed in the singular, the plural is contemplated unless limitation to the singular is explicitly stated.

What is claimed is:

1. A method for determining a severity of motion disorder symptoms based on sensor data, comprising:
receiving, at a processor of a mobile device, the sensor data from one or more sensors;
determining, by the processor, that the sensor data represents one or more activities of daily life (ADLs) of a user;
assigning, by the processor, one or more probabilities to the one or more determined ADLs, each probability of the one or more probabilities indicating a confidence level that the sensor data represents a corresponding ADL of the one or more determined ADLs; and
providing, by the processor, the sensor data and the one or more probabilities to a motion disorder symptom scoring module,
wherein the motion disorder symptom scoring module generates one or more scores for the one or more determined ADLs based on the sensor data, each score of the one or more scores indicating the severity of the motion disorder symptoms for a corresponding ADL of the one or more determined ADLs, and combines the one or more scores and the one or more probabilities to generate an aggregated severity score for the motion disorder symptoms.

2. The method of claim 1, wherein the motion disorder symptom scoring module performs a separate regression analysis of the sensor data corresponding to each ADL of the one or more determined ADLs to determine the severity of the motion disorder symptoms for the corresponding ADL of the one or more determined ADLs.

3. The method of claim 1, wherein the motion disorder symptom scoring module analyzes the sensor data corresponding to each ADL of the one or more determined ADLs based on a category of ADL into which the ADL is classified.

4. The method of claim 1,
wherein the motion disorder symptom scoring module is a component of a second device, and
wherein the sensor data and the one or more probabilities are sent to the motion disorder symptom scoring module over a wireless network.

5. The method of claim 4,
wherein the mobile device comprises a sensor device worn by a user having a motion disorder, and wherein the second device comprises a smartphone, a local hub, or an Internet server.

6. The method of claim 5, wherein the one or more sensors are components of the mobile device.

7. The method of claim 1, wherein the one or more sensors comprise one or more sensors worn by a user of the mobile device having a motion disorder.

8. The method of claim 1, wherein the motion disorder symptom scoring module is a component of the mobile device.

9. The method of claim 8, wherein the mobile device comprises a sensor device worn by a user having a motion disorder.

10. The method of claim 1, wherein the one or more sensors comprise an accelerometer, a gyroscope, a magnetometer, an audio sensor, or any combination thereof.

11. A method of operating a device that is configured to execute a motion disorder symptom scoring module, comprising:
receiving, at the motion disorder symptom scoring module, sensor data that is representative of one or more activities of daily life (ADLs) of a user;
receiving, at the motion disorder symptom scoring module, one or more probabilities that are assigned to the one or more determined ADLs, each probability of the one or more probabilities indicating a confidence level that the sensor data represents a corresponding ADL of the one or more determined ADLs;
generating, by the motion disorder symptom scoring module, one or more scores for the one or more determined ADLs based on the sensor data, each score of the one or more scores indicating the severity of the motion disorder symptoms for a corresponding ADL of the one or more determined ADLs; and
combining, by the motion disorder symptom scoring module, the one or more scores and the one or more probabilities to generate an aggregated severity score for the motion disorder symptoms.

12. The method of claim 11, further comprising:
performing, by the motion disorder symptom scoring module, a separate regression analysis of the sensor data corresponding to each ADL of the one or more determined ADLs to determine the severity of the motion disorder symptoms for the corresponding ADL of the one or more determined ADLs.

13. The method of claim 11, further comprising:
analyzing, by the motion disorder symptom scoring module, the sensor data corresponding to each ADL of the one or more determined ADLs based on a category of ADL into which the ADL is classified.

14. The method of claim 11, wherein the motion disorder symptom scoring module receives the sensor data and the one or more probabilities from a second device over a wireless network.

15. The method of claim 14,
wherein the device comprises a smartphone, a local hub, or an Internet server, and
wherein the second device comprises a sensor device worn by a user having a motion disorder.

16. The method of claim 11, wherein the device comprises a sensor device worn by a user having a motion disorder.

17. An apparatus for determining a severity of motion disorder symptoms based on sensor data, comprising:
at least one processor configured to:
receive the sensor data from one or more sensors;
determine that the sensor data represents one or more activities of daily life (ADLs) of a user;
assign one or more probabilities to the one or more determined ADLs, each probability of the one or more probabilities indicating a confidence level that the sensor data represents a corresponding ADL of the one or more determined ADLs; and
provide the sensor data and the one or more probabilities to a motion disorder symptom scoring module,
wherein the motion disorder symptom scoring module is configured to generate one or more scores for the one or more determined ADLs based on the sensor data, each score of the one or more scores indicating the severity of the motion disorder symptoms for a corresponding ADL of the one or more determined ADLs, and combine the one or more scores and the one or more probabilities to generate an aggregated severity score for the motion disorder symptoms.

18. The apparatus of claim 17, wherein the motion disorder symptom scoring module is configured to perform a separate regression analysis of the sensor data corresponding to each ADL of the one or more determined ADLs to determine the severity of the motion disorder symptoms for the corresponding ADL of the one or more determined ADLs.

19. The apparatus of claim 17, wherein the motion disorder symptom scoring module is configured to analyze the sensor data corresponding to each ADL of the one or more determined ADLs based on a category of ADL into which the ADL is classified.

20. The apparatus of claim 17,
wherein the motion disorder symptom scoring module is a component of a second device, and
wherein the sensor data and the one or more probabilities are sent to the motion disorder symptom scoring module over a wireless network.

21. The apparatus of claim 20,
wherein the apparatus comprises a sensor device worn by a user having a motion disorder, and
wherein the second device comprises a smartphone, a local hub, or an Internet server.

22. The apparatus of claim 17, wherein the one or more sensors comprise one or more sensors worn by a user of the apparatus having a motion disorder.

23. The apparatus of claim 17, wherein the motion disorder symptom scoring module is a component of the apparatus.

24. The apparatus of claim 23, wherein the apparatus comprises a sensor device worn by a user having a motion disorder.

25. An apparatus for operating a device that is configured to execute a motion disorder symptom scoring module, comprising:
at least one processor configured to:
receive sensor data that is representative of one or more activities of daily life (ADLs) of a user;
receive one or more probabilities that are assigned to the one or more determined ADLs, each probability of the one or more probabilities indicating a confidence level that the sensor data represents a corresponding ADL of the one or more determined ADLs;
generate one or more scores for the one or more determined ADLs based on the sensor data, each score of the one or more scores indicating the severity of the motion disorder symptoms for a corresponding ADL of the one or more determined ADLs; and
combine the one or more scores and the one or more probabilities to generate an aggregated severity score for the motion disorder symptoms.

26. The apparatus of claim 25, wherein the at least one processor is further configured to:
perform a separate regression analysis of the sensor data corresponding to each ADL of the one or more determined ADLs to determine the severity of the motion disorder symptoms for the corresponding ADL of the one or more determined ADLs.

27. The apparatus of claim 25, wherein the at least one processor is further configured to:
analyze the sensor data corresponding to each ADL of the one or more determined ADLs based on a category of ADL into which the ADL is classified.

28. The apparatus of claim 25, wherein the at least one processor receives the sensor data and the one or more probabilities from a second device over a wireless network.

29. The apparatus of claim 28,
wherein the device comprises a smartphone, a local hub, or an Internet server, and
wherein the second device comprises a sensor device worn by a user having a motion disorder.

30. The apparatus of claim 25, wherein the device comprises a sensor device worn by a user having a motion disorder.
